(19) United States
(12) Patent Application Publication
(10) Pub. No.: US 2025/0146029 A1
(43) Pub. Date: May 8, 2025

(54) METHOD OF PREPARING IBUPROFEN BY ENZYMATIC CONVERSION AND A MODIFIED POLYPEPTIDE THEREOF

(71) Applicant: Naveen K. Kulkarni, Bengaluru (IN)

(72) Inventor: Naveen K. Kulkarni, Bengaluru (IN)

(21) Appl. No.: 18/386,442

(22) Filed: Nov. 2, 2023

(51) Int. Cl. C12P 7/40; C12N 9/02

(52) U.S. Cl. CPC C12P 7/40; C12N 9/0008; C07K 2319/21

(57) ABSTRACT

The present invention relates to an enzymatic conversion of aldehyde to carboxylic acid for the preparation of ibuprofen. In particular, the present disclosure provides a method of preparation of 2-(4-isobutylphenyl) propanoic acid that is ibuprofen by enzymatic conversion of 2-(4-isobutylphenyl) propanal that is ibuprofen aldehyde to 2-(4-Isobutylphenyl) propanoic acid that is ibuprofen in presence of an oxidoreductase enzyme with high conversion efficiency.

### FIELD OF INVENTION

The present disclosure relates to an enzymatic conversion of aldehyde to carboxylic acid for the preparation of ibuprofen. In particular, the present disclosure provides a green synthesis method of preparation of 2-(4-isobutylphenyl) propanoic acid, that is ibuprofen, by enzymatic conversion of 2-(4-isobutylphenyl) propanal in presence of a modified polypeptide belonging to the class oxidoreductase.

### BACKGROUND OF THE INVENTION

Ibuprofen is a nonsteroidal anti-inflammatory drug (NSAID) which is commonly used to relieve pain, reduce inflammation, and also in subsiding fever. Its synthesis involves several chemical steps and the commercial production of ibuprofen is a complex and multi-step process. Nevertheless, there are no known enzymatic method for the conversion of Ibuprofen aldehyde to Ibuprofen. However, there are chemical routes for this process. The aforementioned conversion is seen in Boot's ibuprofen synthesis which begins with Friedel-Crafts acylation of isobutyl benzene with acetyl chloride followed by converting the para-acetyl group is to ibuprofen's propionic acid substituent in the following steps; (i) The acetyl group is converted to an α, β-epoxy ester via a Darzens reaction with ethyl chloroacetate, (ii) The epoxy ester is hydrolyzed and decarboxylated to give ibuprofen aldehyde, (iii) Aldehyde is oxidized to carboxylic acid using sodium dichromate and dilute sulfuric.

However, the Boots method have several limitations. For instance, the Boot's procedure involves hazardous chemicals in the production process of ibuprofen and formation of undesired side products. Further, the Boots process involves several steps, including oxidation, hydrolysis, cyclization, and multiple chemical transformations. The complexity of the process requires careful control and can make it challenging to optimize and scale up. Also, some of the chemicals and reagents used in the process can be hazardous and process requires proper safety measures and waste disposal procedures are required to ensure worker safety and environmental protection. For instance, as per New Jersey Hazardous for Health, 2009, sodium dichromate can affect dangerously as it is carcinogenic in nature and needs to be handled with care. Nevertheless, when inhaled and passed through the skin, can irritate and burn the skin and eyes with possible eye damage. There are multiple other drawbacks and safety concerns while using sodium dichromate and is not recommended for the safety purpose.

Further, the synthesis process generates chemical by-products and waste materials that must be managed and disposed of properly. This can increase the cost and environmental impact of production. As observed, there are no known enzymatic method for the conversion of Ibuprofen aldehyde to Ibuprofen. As the original synthesis of Ibuprofen generates a lot of dangerous waste and by-products, the energy was consumed with lower yield of the final product, so that it was necessary to improve or to modify the route of Ibuprofen synthesis.

Therefore, there is an unmet need in the art to provide a green chemistry method for preparation of ibuprofen that can overcome one or more disadvantages of the existing chemical synthesis method such as eliminate the side product formation and reduces the need to use of chemicals that are harmful to the environment. Hence, a green chemistry method with new route for Ibuprofen synthesis is the need of the hour with minimum steps of reactions, involving a lower amount of waste and by products, only few intermediate reagents, where most of them can be reused, and a higher atom efficiency than the classical route.

### OBJECTS OF THE INVENTION

An object of the present disclosure is to provide an enzymatic conversion method of preparing 2-(4.-isobutylphenyl) propanoic acid, that is ibuprofen.

An object of the present disclosure is to provide a method for an enzymatic conversion of aldehyde to carboxylic acid for the preparation of ibuprofen.

An object of the present disclosure is to provide an engineered oxidoreductase polypeptide having improved properties as compared to a naturally occurring wild-type oxidoreductase enzyme.

An object of the present disclosure is to provide a green chemistry method to prepare the ibuprofen by enzymatic conversion which does not produce hazardous chemicals.

An object of the present disclosure is to provide an easy and rapid method for preparing ibuprofen.

### SUMMARY OF THE INVENTION

The present invention provides a method for an enzymatic conversion of aldehyde to carboxylic acid for the preparation of ibuprofen. The present disclosure also provides an engineered oxidoreductase polypeptide having improved properties as compared to a naturally occurring wild-type oxidoreductase enzyme

A general aspect of the present disclosure, present invention provides a method of preparation of alkyl substituted phenyl carboxylic acids of the formula I using the alkyl substituted phenyl aldehydes of the formula II as substrate using an oxidoreductase enzyme, a cofactor and a buffer.

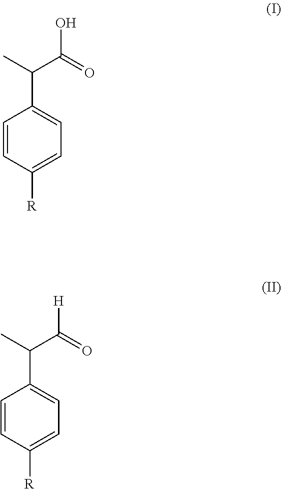

wherein R is an alkyl group of the formula CₙH₂ₙ₊₁ with n=0-4 and R-group substitutions can be hydrogen, methyl, ethyl, propyl, isopropyl, butyl, tert-butyl, isobutyl.

In another embodiment, the present invention provides a method of preparation of 2-(4-isobutylphenyl) propanoic acid by enzymatic conversion of 2-(4-isobutylphenyl) propanal to 2-(4-isobutylphenyl) propanoic acid by an oxidoreductase enzyme in the presence of a buffer and a cofactor.

In yet another embodiment, said buffer solution is a mixture of monobasic and dibasic potassium phosphates.

In still another embodiment, the ratio range of the enzyme: substrate is 1:1 or 2:1 or 4:1

In yet another embodiment, said cofactor is an oxidized form of nicotinamide adenine dinucleotide (NAD+), and wherein said cofactor is present in a concentration ranging from 0.01 mM to 10 mM, preferably 2 mM to 5 mM of the reaction mixture.

In still another embodiment, said oxidoreductase enzyme is selected from an aldehyde dehydrogenase enzyme family.

In yet another embodiment, said aldehyde dehydrogenase enzyme comprises an amino acid sequence having at least 30% homology with amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

In still another embodiment, said aldehyde dehydrogenase enzyme is a recombinant enzyme produced as a fusion protein with 6× His tag and is selected from in a completely purified state, in a partially purified state, or in the microbial cells in which it is expressed.

In yet another embodiment, said cells are bacterial cells in a native state, or a lysed state.

In another embodiment, the present invention discloses a modified oxidoreductase polypeptide comprising the SEQ ID NO: 11, SEQ ID NO: 12, SEQ ID NO: 13, SEQ ID NO: 14, and/or SEQ ID NO: 15 wherein said recombinant polypeptides comprise of amino acid substitutions from the group consisting of;
i. Asp85 substituted with Arg or any polar, aliphatic, basic amino acid;
ii. Glu483 substituted with Arg or any polar, aliphatic, basic amino acid;
iii. Asp490 substituted with Arg or any polar, aliphatic, basic amino acid;
iv. Glu494 substituted with Arg or any polar, aliphatic, basic amino acid.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE ACCOMPANYING DRAWINGS

The following drawings form part of the present specification and are included to further illustrate aspects of the present disclosure. The disclosure may be better understood by reference to the drawings in combination with the detailed description of the specific embodiments presented herein.

FIG. 1A illustrates a diagrammatical representation showing the distance between carbonyl carbon of substrate and C4N of NAD.

FIG. 1B illustrates a diagrammatical representation showing the distance between carbonyl carbon of substrate and catalytic cysteine.

FIG. 2 illustrates a thin layer chromatography (TLC) image showing conversion with different aldehyde dehydrogenase enzymes having amino acid sequence Seq ID NO:1, Seq ID NO: 2, Seq ID NO:3, Seq ID NO:4 and Seq ID NO:5; wherein 101: Substrate, 102: Product, Lane 1: Substrate standard; Lane 2: Product standard; Lane 3: Substrate blank; Lane 4: Product blank; Lane 5: Reaction with Seq ID NO:1, Lane 6: Reaction with Seq ID NO:2, Lane 7: Reaction with Seq ID NO:3, Lane 8: Reaction with Seq ID NO:4 and Lane 9: Reaction with Seq ID NO:5.

FIG. 3A illustrates a RP-HPLC chromatogram of a substrate standard.

FIG. 3B illustrates a RP-HPLC chromatogram of a product standard.

FIG. 3C illustrates a RP-HPLC chromatogram of a reaction with Seq ID NO:1.

FIG. 3D illustrates a RP-HPLC chromatogram of a reaction with Seq ID NO:2.

FIG. 3E illustrates a RP-HPLC chromatogram of a reaction with Seq ID NO:3.

FIG. 3F illustrates a RP-HPLC chromatogram of a reaction with Seq ID NO:4.

FIG. 3G illustrates a RP-HPLC chromatogram of a reaction with Seq ID NO:5.

FIG. 4 illustrates a thin layer chromatography (TLC) image showing conversion of 2-(4-isobutylphenyl) propanal to 2-(4-Isobutylphenyl) propanoic acid using aldehyde dehydrogenase enzyme having amino acid sequence Seq ID NO:1 in a 300 mL reaction scale, wherein 101: Substrate, 102: Product, Lane 1: Substrate and Product standard; Lane 2: Reaction with Seq ID NO:1.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability.

All methods described herein can be performed in suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The headings and abstract of the invention provided herein are for convenience only and do not interpret the scope or meaning of the embodiments.

Various terms are used herein. To the extent a term used in a claim is not defined below, it should be given the broadest definition persons in the pertinent art have given that term as reflected in printed publications and issued patents at the time of filing.

The problem solved by the present invention is a greener approach for the production of ibuprofen. This invention mainly focuses on the process of conversion of aldehyde to carboxylic acid for production of ibuprofen i.e., through oxidation of the aldehyde group. This has been solved by using enzyme family oxidoreductase. The enzymes form oxidoreductase family are enzymes that can catalyze oxidation or reduction reaction. The enzyme chosen in this invention is a subclass of oxidoreductase family which is aldehyde dehydrogenase. These enzymes catalyze the conversion of aldehyde group to its corresponding carboxylic acid, coupled with the utilization of NAD+ as the cofactor.

The present invention eliminates the side product formation and also reduces the need to use chemicals that are harmful to the environment. More specifically, this invention discloses the enzymes belonging to the aldehyde dehydrogenase that oxidizes the aldehyde to carboxylic acid.

A general aspect of the present disclosure, present invention provides a method of preparation of alkyl substituted phenyl carboxylic acids of the formula I using the alkyl substituted phenyl aldehydes of the formula II as substrate using an oxidoreductase enzyme, a cofactor and a buffer.

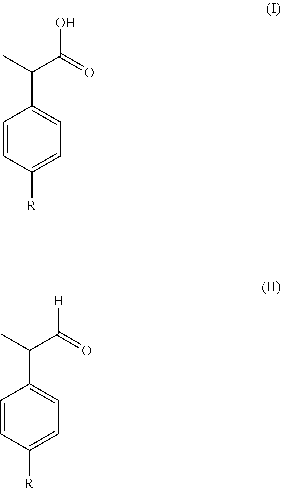

wherein R is an alkyl group of the formula CₙH₂ₙ₊₁ with n=0-4 and R-group substitutions can be hydrogen, methyl, ethyl, propyl, isopropyl, butyl, tert-butyl, isobutyl.

In another embodiment, the present invention provides a method of preparation of 2-(4-isobutylphenyl) propanoic acid by enzymatic conversion of 2-(4-isobutylphenyl) propanal to 2-(4-isobutylphenyl) propanoic acid by an oxidoreductase enzyme in the presence of a buffer and a cofactor.

In yet another embodiment, said buffer solution is a mixture of monobasic and dibasic potassium phosphates.

In still another embodiment, the ratio range of the enzyme: substrate is 1:1 or 2:1 or 4:1.

In yet another embodiment, said cofactor is an oxidized form of nicotinamide adenine dinucleotide (NAD+), and wherein said cofactor is present in a concentration ranging from about 0.01 mM to 10 mM, preferably 2 mM to 5 mM the reaction mixture.

In still another embodiment, said oxidoreductase enzyme is selected from an aldehyde dehydrogenase enzyme.

In yet another embodiment, said aldehyde dehydrogenase enzyme comprises an amino acid sequence having at least 30% homology with amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

In still another embodiment, said aldehyde dehydrogenase enzyme is a recombinant enzyme produces as a fusion protein with 6× His tag and is selected from in a completely purified state, in a partially purified state, or in the microbial cells in which it is expressed.

In yet another embodiment, said cells are bacterial cells in a native state, or a lysed state.

In another embodiment, the present invention discloses a modified oxidoreductase polypeptide comprising the SEQ ID NO: 11, SEQ ID NO: 12, SEQ ID NO: 13, SEQ ID NO: 14, and/or SEQ ID NO: 15 wherein said recombinant polypeptides comprise of amino acid substitutions from the group consisting of;
i. Asp85 substituted with Arg or any polar, aliphatic, basic amino acid;
ii. Glu483 substituted with Arg or any polar, aliphatic, basic amino acid;
iii. Asp490 substituted with Arg or any polar, aliphatic, basic amino acid;
iv. Glu494 substituted with Arg or any polar, aliphatic, basic amino acid.

The conversion of 2-(4-isobutylphenyl) propanal to 2-(4-Isobutylphenyl) propanoic acid may be performed using an aldehyde dehydrogenase enzyme selected from in a completely purified state, in a partially purified state, or in the microbial cells in which it was expressed. The cells themselves may be in a native state or a lysed state. It will be appreciated by those of ordinary skill in the art that use of the enzyme in the cells is preferred for the practice of the process of the invention since it represents a significant savings in cost.

Preferably, the enzyme is expressed in E. coli and used as a suspension of native cells. More preferably, the enzyme is used as a lysate of cells.

The organism producing the polypeptides having aldehyde dehydrogenase activity useful in the enzymatic conversion of aldehyde to carboxylic acid may be a wild type strain or a variant and is preferably selected from bacteria belonging to the Family Burkholderiaceae or Enterobacteriaceae or Thermaceae or Rhizobiaceae or Pseudomonadaceae or Bacillaceae. The method of preparation 2-(4-isobutylphenyl) propanoic acid by enzymatic conversion of 2-(4-isobutylphenyl) propanal to 2-(4-Isobutylphenyl) propanoic acid using aldehyde dehydrogenase enzyme is coupled with a cofactor.

In the reaction, 2-(4-isobutylphenyl) propanal acts as a substrate. The amount of 2-(4-isobutylphenyl) propanal substrate in the reaction mixture is preferably greater than about 0.1% by weight and may be increased to about 50% by weight, with a preferred concentration being from about 0.2 to 5% by weight.

The amount of aldehyde dehydrogenase enzyme can be loaded at the concentration of at least 5% to about 100%. The enzyme concentration for loading may be from about 10% to about 100%, which may be 10%, 15%, 20%, 25%, 30%, 35%, 40%, 45%, 50%, 55%, 60%, 65%, 70%, 75%, 80%, 85%, 90%, 95% or 100%.

The cofactor is present in the reaction mixture in a concentration from about 0.01 mM to 10 mM, preferably 2 mM to 5 mM.

The enzymatic conversion method of the present invention can be advantageously carried out in continuous or batch mode production.

The enzymatic conversion method of the present invention is non-polluting and sustainable to the environment. The method used here for the production of ibuprofen also ensures high purity, without the formation of undesirable byproducts. The present invention provides a novel method for the conversion of 2-(4-Isobutylphenyl) propanal (ibuprofen aldehyde) to 2-(4-Isobutylphenyl) propanoic acid (ibuprofen). The conversion from ibuprofen aldehyde to ibuprofen is a part of the chemical RoS. This invention involves the use of an enzyme, which is novel for this procedure. This process involves the oxidation of the aldehyde group into carboxylic group with the help of an aldehyde dehydrogenase enzyme.

Although the subject matter has been described in considerable detail with reference to certain preferred embodiments thereof, other embodiments are possible.

The present disclosure satisfies the existing needs, as well as others, and generally overcomes the deficiencies found in the existing art.

### Advantages of the Present Invention

The present disclosure provided an efficient method of preparation 2-(4-isobutylphenyl) propanoic acid that is ibuprofen by enzymatic conversion of 2-(4-isobutylphenyl) propanal that is ibuprofen aldehyde to 2-(4-Isobutylphenyl) propanoic acid that is ibuprofen in presence of enzyme.

The method for preparation of ibuprofen in accordance with the present invention by enzymatic conversion overcomes the disadvantages of the existing chemical synthesis method by reducing the need to use of chemicals that are harmful to the environment and human beings.

The enzymatic conversion method of the present invention is a green method and a non-polluting and sustainable to the environment.

The method used here for the production of ibuprofen also ensures high purity, without the formation of undesirable byproducts.

The enzymatic conversion method of the present invention can be advantageously carried out in continuous or batch mode production.

The enzymatic conversion method of the present invention provides higher conversion rate of substrate to end product.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Example 1 Wild-Type Aldehyde Dehydrogenase Gene Acquisition and Construction of Expression Vectors

Aldehyde dehydrogenase encoding genes were designed for recombinant expression in Escherichia coli based on the reported amino acid sequence of the aldehyde dehydrogenase from a nucleotide database and a codon optimization algorithm. Genes were synthesized and cloned into expression vector pET28a under the control of a T7 promoter. The expression vector contained the pBR322 origin of replication and the kanamycin resistance gene. Resulting plasmids were transformed into E. coli DH5a using standard methods and stocks of plasmids were prepared using alkaline lysis method for future use.

### Example 2 Production of Aldehyde Dehydrogenase-Shake Flask Procedure

The plasmids containing aldehyde dehydrogenase genes were transformed into E. coli BL21 (DE3) expression strain using standard methods. A single microbial colony of E. coli expressing the plasmid containing the aldehyde dehydrogenase gene was inoculated into 5 mL Luria Bertani Broth containing 20 μg/ml kanamycin. The bacterial culture was grown overnight (at least 12 hrs) in an incubator at 37° C. with continuous shaking at 200 rpm. The culture was used as primary inoculum at 1-2% to inoculate 400 ml Terrific Broth (10 g/L tryptone, 22 g/L yeast extract, 4 ml/L glycerol. 72 mM dipotassium hydrogen phosphate, 17 mM monopotassium dihydrogen phosphate) in 1-liter flask containing 20 μg/ml kanamycin. The bacterial culture was grown in an incubator at 37° C. with continuous shaking at 200 rpm. Expression of the gene of interest was induced with 0.6 mM IPTG when the OD600 of the culture is 0.6 to 0.8 and incubated overnight (at least 16 hrs). Cells were harvested by centrifugation (6000 rpm, 5 min) and the spent media supernatant was discarded. The cell pellet was resuspended with ice cold 50 mM Tris-Cl buffer containing 100 mM NaCl, pH 7.5, at 1:10 ratio (cell pellet: buffer) and lysed by sonication by maintaining the samples at 4° C. Cell debris was removed by centrifugation (10000 rpm, 45 min. 4° C.). The clear lysate supernatant was collected and stored at −20° C. Total protein of the lysate was estimated using Bradford method and the quality of lysate was checked using 12% SDS-PAGE.

### Example 3 Evaluation of the Aldehyde Dehydrogenase Enzymes for Oxidation of 2-(4-Isobutylphenyl) Propanal to 2-(4-Isobutylphenyl) Propanoic Acid

Different aldehyde dehydrogenase enzymes were screened for the oxidation of 2-(4-isobutylphenyl) propanal to 2-(4-isobutylphenyl) propanoic acid. The assay was performed with a total reaction mixture of 1.0 ml in a 2.0 ml vial. The reaction was carried out with a substrate: enzyme ratio of 1:1 or 2:1 or 4:1. Potassium phosphate buffer (pH-9) along with NAD+ cofactor was added to the vial followed by enzyme and mixed for 5 minutes. The substrate resuspended in 100% DMSO was added in drops. The assay mixture was incubated at 40° C. for 24-30 h under continuous agitation. After 24-30 h, the reaction was quenched and the final product was extracted from the reaction mixture by adding ethyl acetate in 2:1 ratio (solvent: reaction mixture). The extraction was repeated twice and the organic layer was collected by centrifugation. The organic layer was concentrated using a rotary evaporator. The concentrated sample was subjected to analysis using RP-HPLC as per the protocol provided in Example 4.

### Example 4 Analytical Method for Conversion of 2-(4-Isobutylphenyl) Propanal to 2-(4-Isobutylphenyl) Propanoic Acid

| Sample Preparation | The dried sample after evaporation of solvent | |
|---|---|---|
| | from the concentrated organic layer was | |
| | resuspended in 1 ml of the mobile phase and | |
| | filtered using 0.2μ membrane filter | |
| Column | Inertsil ODS-3V (4.6 × 250 mm), 5 μm or | |
| | equivalent | |
| Mobile phase | 600 volumes of Acetonitrile + 400 volume | |
| | of water + 1.0 mL of Ortho phosphoric acid | |
| Diluent for sample | Mobile phase | |
| preparation | | |
| Injection volume | 10 | μL |
| Detection | Detection at 214 nm | |
| Flow rate | 1.5 | mL/min |
| Column temperature | 25° | C. |
| Total Run time/ sample | 30 | min |

### Example 5 Methodology of Generating Modified Recombinant Polypeptides

An in-silico enzyme engineering framework was used (QZyme Workbench™) to engineer the aldehyde dehydrogenase enzyme to achieve high catalytic efficiency. The framework carries out different aspects of in silico protein engineering including structural refinement and modelling, ligand docking, conformational sampling, estimating substrate binding affinity, modelling catalytic reaction, identifying mutable hotspots, further hotspot optimization.

The three-dimensional structure of the protein (aldehyde dehydrogenase) was modelled. Additional structural refinement was carried out to ensure that the modelled structure satisfied catalytically competent conformation (open vs closed state). The modelled structure thus obtained was used to model the near-attack conformation of the substrate in the enzyme active site (Michaelis complex) by implementing several docking algorithms. In this step, the bottle neck of the enzymatic conversion of the substrate was also determined. In particular, the three areas were investigated in detail:
a) Dynamics of Michaelis complex through classical molecular dynamics simulation to assess the stability of Michaelis complex;
b) Non-equilibrium molecular dynamics simulation to study the substrate entry, product exit and to estimate associated free energy barriers; and
c) The rate-limiting step of the reaction using hybrid quantum mechanics/molecular mechanics (QM/MM) approach. The energy barriers associated with each step are further compared to identify the step that has the highest activation barrier (rate-limiting).

To address the bottleneck of enzymatic conversion identified in the previous step, the next step included discovery of key functional residues and evaluation of their mutability in an effort to improve the enzyme function. The step further incorporated a rapid screening method to know the contribution of each amino acid and all possible amino acid substitutions to the enzyme's function and stability. Sequence analysis as well as contact score analysis was given priority for selection of hotspots. Hotspots were selected based on partial conserved residues obtained through Delta-BLAST using NR database. The conserved residues obtained through contact score analysis was selected. All the conserved residues were neglected whereas the partially conserved residues were checked to create a focused library. Apart from sequence alignment, stability analysis was carried out for selecting hotspots. The common mutations obtained through sequence alignment, and stability analysis were shortlisted for the designing step. Thus, a huge library of variants is created, followed by the creation of a focused library.

In the designing step, the rate-limiting step (previously referred to as the bottleneck) was modelled for each of the variants belonging to the focused library, and compared with the wild type enzyme. In addition to that, binding energy calculations were then performed for all the variants from the focused library. The purpose of the designing step is to reduce the false positives and to increase the quality of the focused library. As a final outcome, top variants were shortlisted for further validation through lab experiments.

TABLE 1
Lists of the Mutations in Aldehyde dehydrogenase enzyme:

| S. No | Sequence ID | Mutations |
|---|---|---|
| 1 | SEQ ID NO: 11 | Asp85Arg, Glu483Arg |
| 2 | SEQ ID NO: 12 | Asp85Arg |
| 3 | SEQ ID NO: 13 | Glu483Arg |
| 4 | SEQ ID NO: 14 | Asp490Arg |
| 5 | SEQ ID NO: 15 | Glu494Arg |

TABLE 2
Percentage similarity and relative activity of the different enzymes:

| | Percentage similarity | Relative activity to Seq ID |
|---|---|---|
| Seq ID | to Seq ID No: 1 | NO: 1 |
| 1 | 100 | ++ |
| 2 | 33 | ++ |
| 3 | 32 | ++ |
| 4 | 34 | ++ |
| 5 | 34 | ++ |
| 6 | 35 | + |
| 7 | 46 | + |
| 8 | 36 | + |
| 9 | 30 | + |
| 10 | 35 | + |
| 11 | 99 | ++++ |
| 12 | 99 | ++++ |
| 13 | 99 | +++ |
| 14 | 99 | +++ |
| 15 | 99 | +++ |

### Example 6 Oxidation of 2-(4-Isobutylphenyl) Propanal to 2-(4-Isobutylphenyl) Propanoic Acid Using Aldehyde Dehydrogenase

The reactor containing the 3-neck flask of volume 1 liter and temperature controller was used for the oxidation of 2-(4-isobutylphenyl) propanal to 2-(4-isobutylphenyl) propanoic acid. The reaction was carried out with a substrate: enzyme ratio of 2:1. To the flask, 15 mL of 1 M potassium phosphate buffer (pH-9) along with 1.5 mL of 100 mM NAD+ cofactor was added followed by enzyme lysate and mixed by overhead stirring of 100 rpm for 5 minutes. The substrate resuspended in 100% DMSO was added in drops over a period of 5-10 minutes under continuous stirring at 300 rpm. The assay mixture was incubated at 40° C. for 24 h under continuous agitation. After 24 h, the reaction was quenched and the final product was extracted from the reaction mixture by adding ethyl acetate in 2:1 ratio (solvent: reaction mixture). The extraction was repeated twice and the organic layer was collected by centrifugation. The organic layer was concentrated using a rotary evaporator.

The concentrated sample was subjected to analysis using RP-HPLC as per the protocol provided in Example 4.

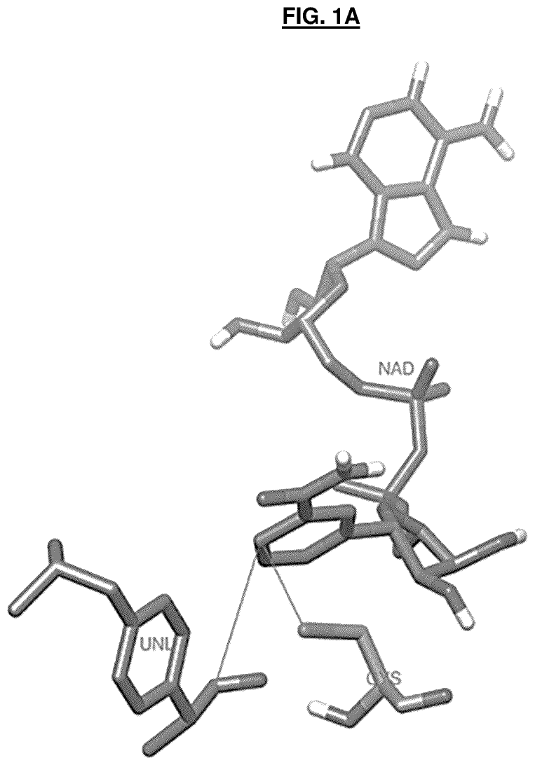

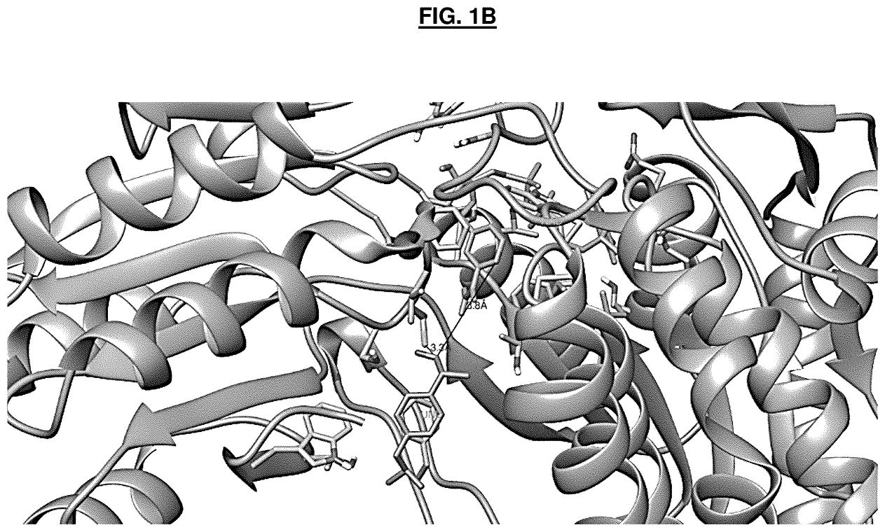

FIG. 1A illustrates a diagrammatical representation showing the distance between carbonyl carbon of substrate and C4N of NAD.

FIG. 1B illustrates a diagrammatical representation showing the distance between carbonyl carbon of substrate and catalytic cysteine.

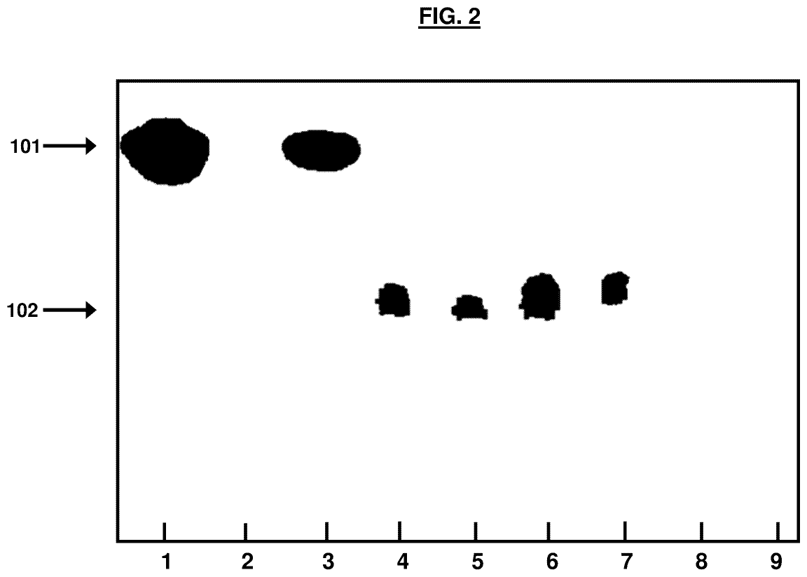

FIG. 2 illustrates a thin layer chromatography (TLC) image showing conversion with different aldehyde dehydrogenase enzymes having amino acid sequence Seq ID NO:1, Seq ID NO: 2, Seq ID NO:3, Seq ID NO:4 and Seq ID NO:5; wherein 101: Substrate, 102: Product, Lane 1: Substrate standard; Lane 2: Product standard; Lane 3: Substrate blank; Lane 4: Product blank; Lane 5: Reaction with Seq ID NO:1, Lane 6: Reaction with Seq ID NO:2, Lane 7: Reaction with Seq ID NO:3, Lane 8: Reaction with Seq ID NO:4 and Lane 9: Reaction with Seq ID NO:5.

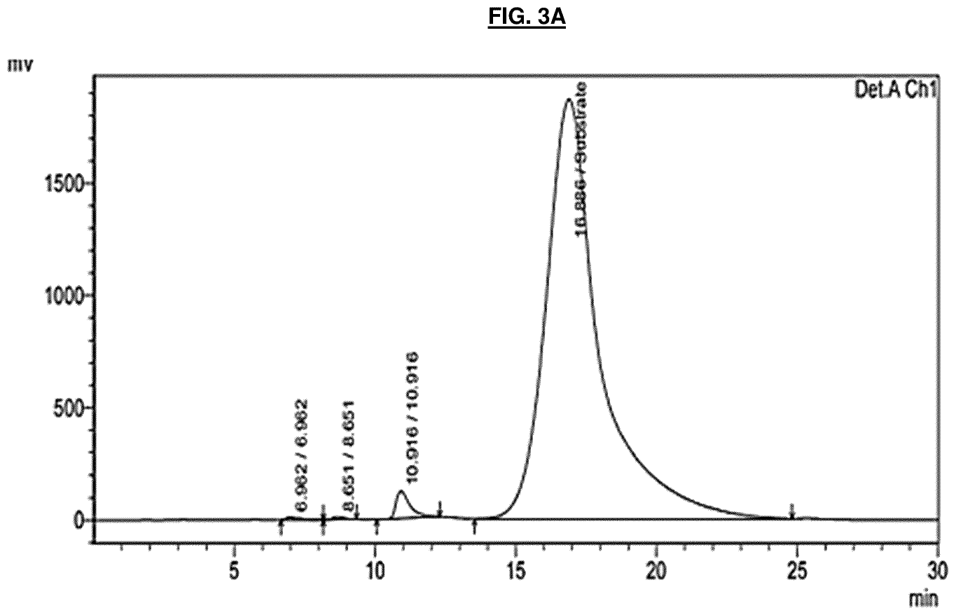

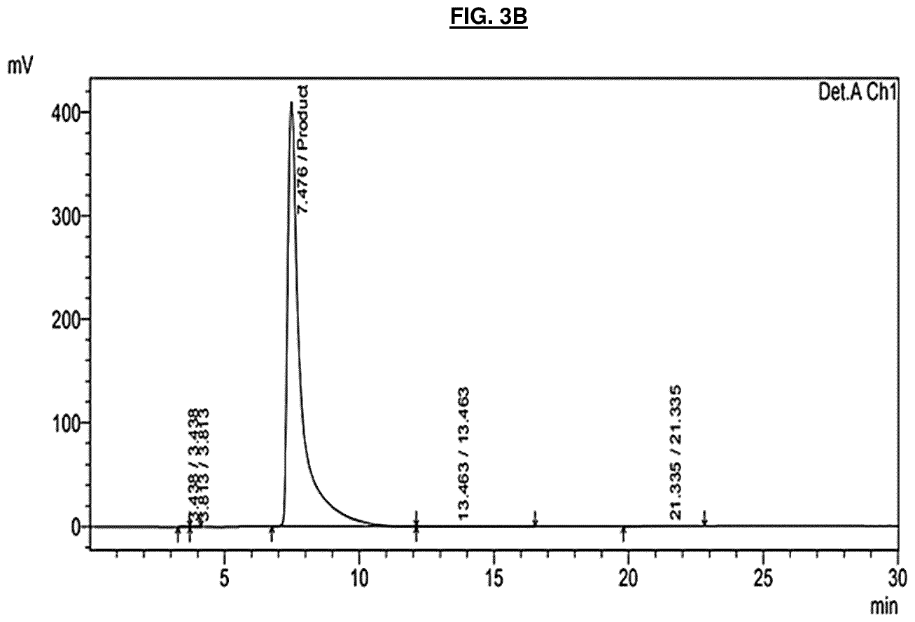

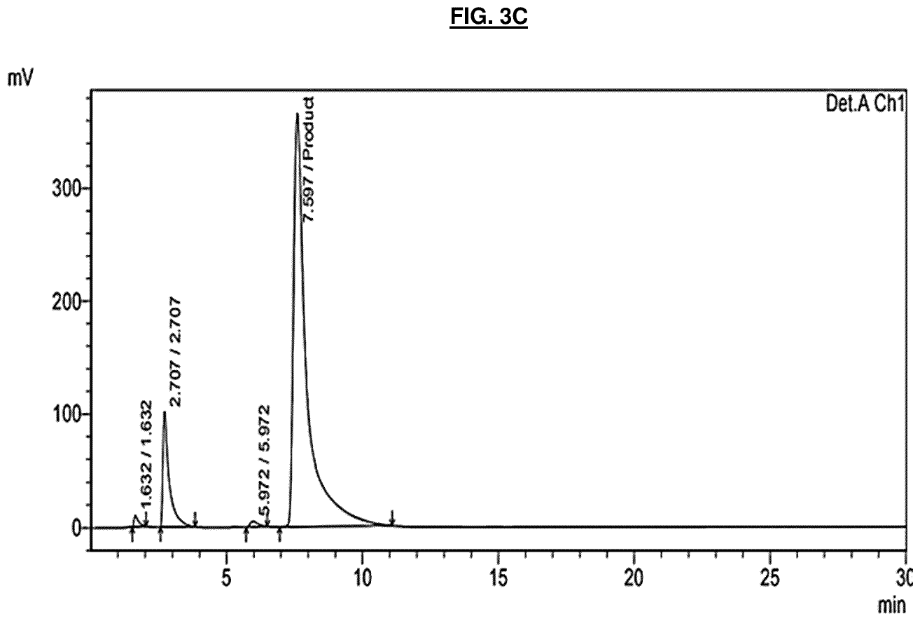

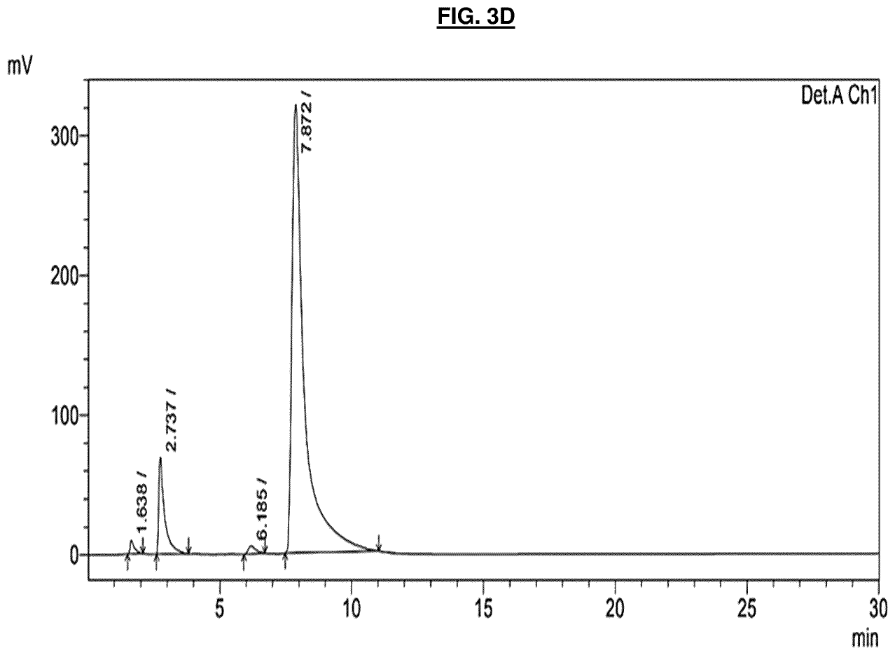

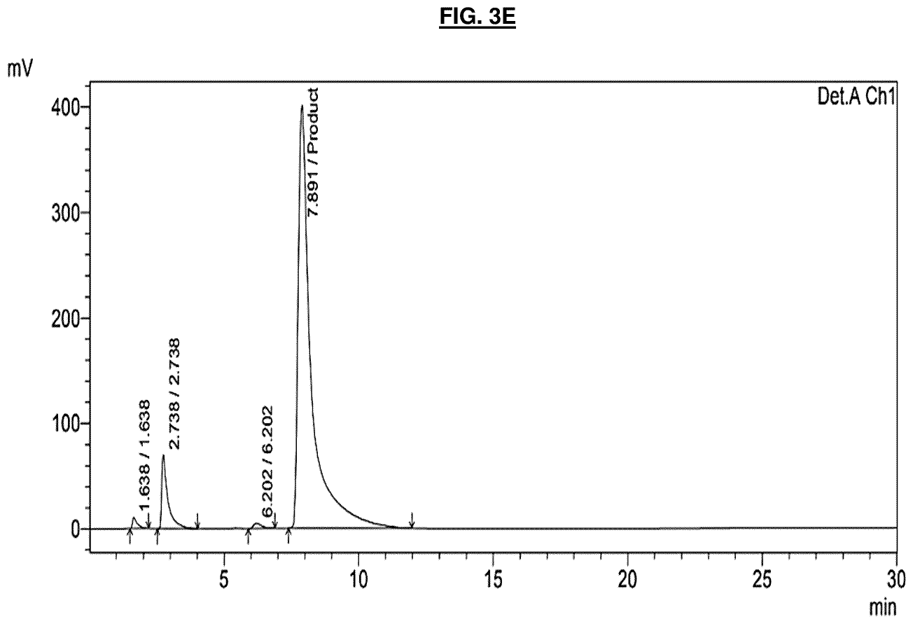

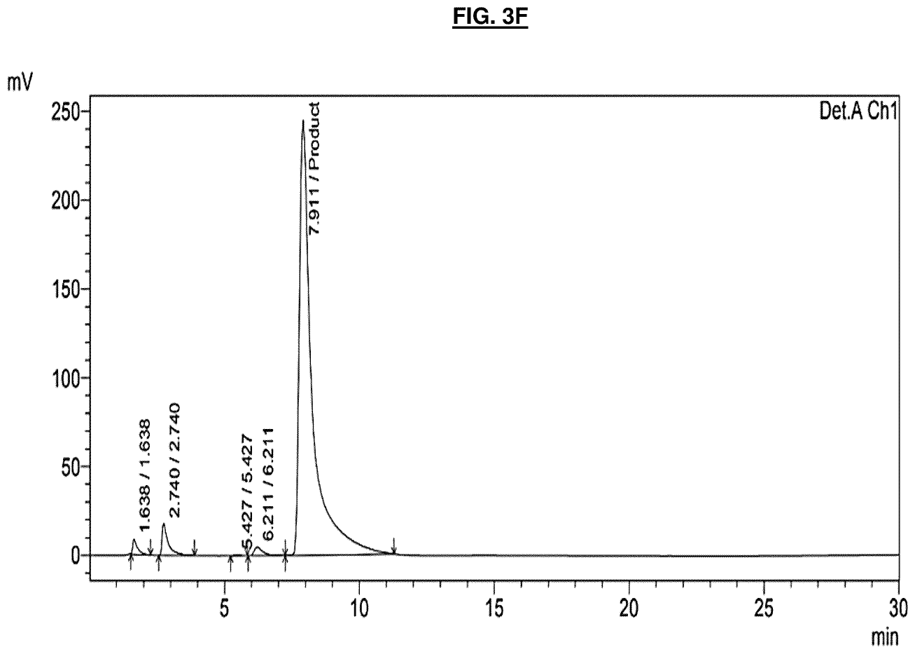

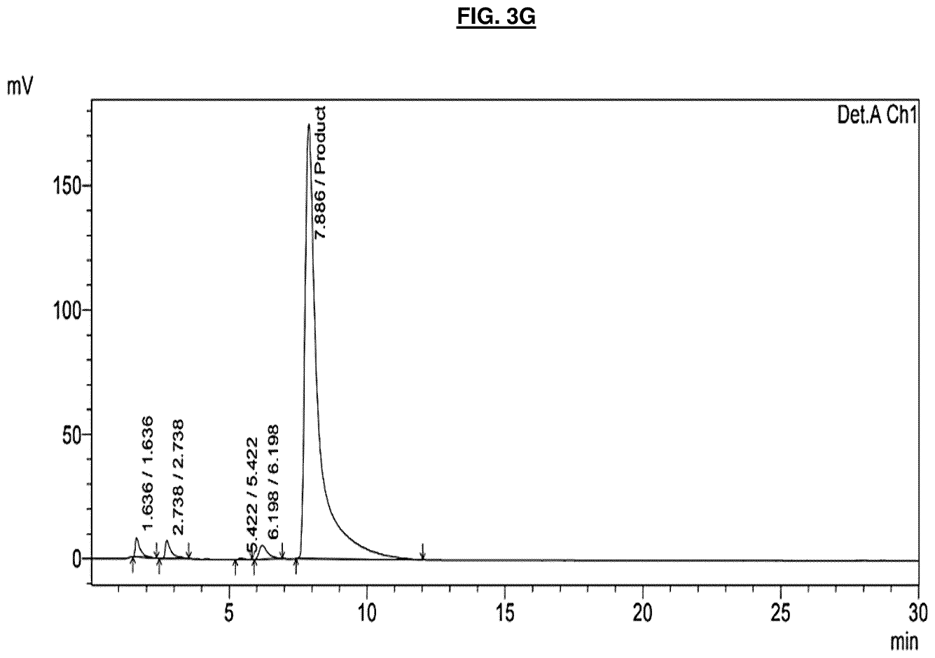

FIG. 3A illustrates a RP-HPLC chromatogram of a substrate standard.

FIG. 3B illustrates a RP-HPLC chromatogram of a product standard.

FIG. 3C illustrates a RP-HPLC chromatogram of a reaction with Seq ID NO:1.

FIG. 3D illustrates a RP-HPLC chromatogram of a reaction with Seq ID NO:2.

FIG. 3E illustrates a RP-HPLC chromatogram of a reaction with Seq ID NO:3.

FIG. 3F illustrates a RP-HPLC chromatogram of a reaction with Seq ID NO:4.

FIG. 3G illustrates a RP-HPLC chromatogram of a reaction with Seq ID NO:5.

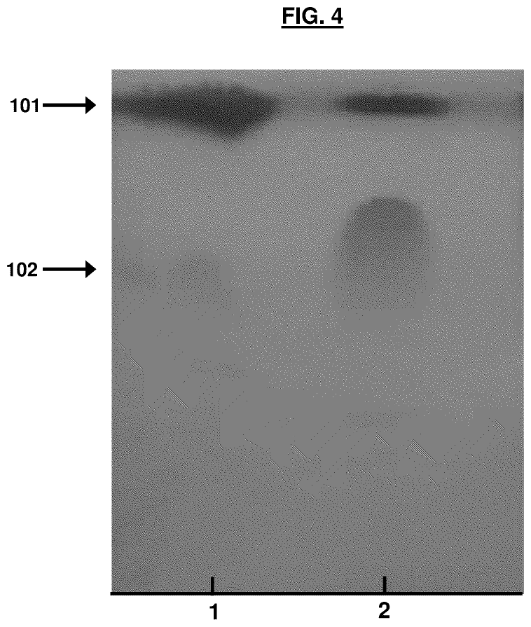

FIG. 4 illustrates a thin layer chromatography (TLC) image showing conversion of 2-(4-isobutylphenyl) propanal to 2-(4-Isobutylphenyl) propanoic acid using aldehyde dehydrogenase enzyme having amino acid sequence Seq ID NO:1 in a 300 mL reaction scale, wherein 101: Substrate, 102: Product, Lane 1: Substrate and Product standard; Lane 2: Reaction with Seq ID NO:1.

Overall, the present invention provides a novel and inventive method for production of ibuprofen by enzymatic conversion which is superior to methods known in the art, and overcomes one or more limitations of the existing chemical synthesis methods.

From the foregoing, it will be appreciated that, although specific embodiments of the invention have been described herein merely for purposes of illustration, various modifications may be made without deviating from the spirit and scope of the invention and should not be construed so as to limit the scope of the invention or the appended claims in any way.

### REFERENCE TO AN ELECTRONIC SEQUENCE LISTING

The contents of the electronic sequence listing (TKH1055_SequenceListing_11022023.xml; Size: 36,206 bytes; and Date of Creation: Nov. 2, 2023) is herein incorporated by reference in its entirety.

What is claimed is:

1. A method of preparation of 2-(4-isobutylphenyl) propanoic acid by enzymatic conversion of 2-(4-isobutylphenyl) propanal to 2-(4-isobutylphenyl) propanoic acid by an oxidoreductase enzyme in the presence of a buffer.

2. A method of preparation of alkyl substituted phenyl carboxylic acids of the formula I using the alkyl substituted phenyl aldehydes of the formula II as substrate using an oxidoreductase enzyme, a cofactor and a buffer. 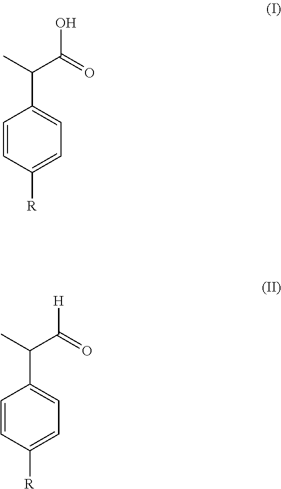
wherein R is an alkyl group of the formula CₙH₂ₙ₊₁ with n=0-4 and R-group substitutions can be hydrogen, methyl, ethyl, propyl, isopropyl, butyl, tert-butyl, isobutyl.

3. The method as claimed in claim 1 wherein, said buffer solution is a mixture of monobasic and dibasic potassium phosphates.

4. The method as claimed in claim 1, wherein the ratio range of the enzyme: substrate is 1:1 or 2:1 or 4:1.

5. The method as claimed in claim 1, wherein said cofactor is an oxidized form of nicotinamide adenine dinucleotide (NAD+).

6. The method as claimed in claim 1, wherein the said oxidoreductase enzyme is selected from an aldehyde dehydrogenase enzyme.

7. The method as claimed in claim 1, wherein said aldehyde dehydrogenase enzyme is selected from the bacteria belonging to the family Burkholderiaceae or Enterobacteriaceae or Thermaceae or Rhizobiaceae or Pseudomonadaceae or Bacillaceae

8. The method as claimed in claim 1, wherein said aldehyde dehydrogenase enzyme comprises an amino acid sequence having at least 30% homology with amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

9. The method as claimed in claim 1, wherein said aldehyde dehydrogenase enzyme is a recombinant enzyme produced as a fusion protein with 6× His tag and is selected from in a completely purified state, in a partially purified state, or in the microbial cells in which it is expressed.

10. The method as claimed in claim 1, wherein said host cells are bacterial cells in a native state, or a lysed state.

11. The method as claimed in claim 10, wherein said host cells are recombinant microorganisms transformed with a nucleic acid construct encoding for the said aldehyde dehydrogenase enzymes.

12. The method as claimed in claim 2, wherein the ratio range of the enzyme: substrate is 1:1 or 2:1 or 4:1.

13. The method as claimed in claim 2 wherein said cofactor is an oxidized form of nicotinamide adenine dinucleotide (NAD+).

14. The method as claimed in claim 2, wherein the said oxidoreductase enzyme is selected from an aldehyde dehydrogenase enzyme.

15. The method as claimed in claim 2, wherein said aldehyde dehydrogenase enzyme is selected from the bacteria belonging to the family Burkholderiaceae or Enterobacteriaceae or Thermaceae or Rhizobiaceae or Pseudomonadaceae or Bacillaceae

16. The method as claimed in claim 2, wherein said aldehyde dehydrogenase enzyme comprises an amino acid sequence having at least 30% homology with amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

17. The method as claimed in claim 2, wherein said aldehyde dehydrogenase enzyme is a recombinant enzyme produced as a fusion protein with 6× His tag and is selected from in a completely purified state, in a partially purified state, or in the microbial cells in which it is expressed.

18. The method as claimed in claim 2, wherein said host cells are bacterial cells in a native state, or a lysed state.

19. An expression vector comprising the polynucleotides encoding for the said aldehyde dehydrogenase enzymes produced by the method as claimed in claim 8.

20. The expression vector as claimed in claim 19, wherein said host cell is a bacterial cell.

21. A modified oxidoreductase polypeptide comprising the SEQ ID NO: 11, SEQ ID NO: 12, SEQ ID NO: 13, SEQ ID NO: 14, and/or SEQ ID NO: 15, wherein the recombinant polypeptides comprise amino acid substitutions from the group consisting of:
a. Asp85 substituted with Arg or any polar, aliphatic, basic amino acid.
b. Glu483 substituted with Arg or any polar, aliphatic, basic amino acid.
c. Asp490 substituted with Arg or any polar, aliphatic, basic amino acid.
d. Glu494 substituted with Arg or any polar, aliphatic, basic amino acid.


SEQUENCE LISTING (15 sequences)

SEQ ID NO: 1 - protein, 1509 aa, Burkholderia cenocepacia
METASNMETGLUTHRASNGLUTHRPHEALALEULEUASPALATHRARGALAPHELEUALALYSPROLYSGLNMETLEUILEGLYALAGLUTRPSERASPALAALASERGLYARGGLNLEUASPVALVALASNPROALAASPGLYTHRVALILEALAARGVALPROGLUALAASPGLUARGASPVALGLNGLNALAVALALAALAALAARGARGALAPHEASPALAGLYPROTRPARGTHRALALYSTHRTHRASPARGGLUARGLEUMETLEUVALLEUALAASPLEUILEGLUALAASNALAARGGLULEUALAGLUILEGLUSERLEUASPASNGLYLYSPROVALMETVALALAGLNGLYLEUASPVALALAMETALAALAGLNCYSPHEARGTYRMETALAGLYTRPALATHRLYSILEGLUGLYSERVALILEASPALAGLYMETPROTYRLEUPROASPSERGLUILEPHEALATYRTHRARGLYSGLUPROVALGLYVALVALGLYALAILEILEPROTRPASNPHEPROLEULEUMETALAALATRPLYSILEALAPROALALEUALATHRGLYCYSTHRVALVALLEULYSPROALAGLUASPTHRPROLEUSERALALEUARGLEUGLYGLULEUILEGLNALAALAGLYPHEPROASPGLYVALVALASNILEVALTHRGLYTYRGLYHISTHRALAGLYALAALALEUSERARGASPPROARGILEASPLYSILEALAPHETHRGLYSERTHRGLNTHRGLYLYSTHRILEGLYHISALAALALEUASPASNMETTHRARGMETSERLEUGLULEUGLYGLYLYSSERPROVALILEVALLEUPROASPVALASPLEUASPLYSALAALAGLNGLYVALALAASNALAILEPHEPHEASNGLNGLYGLNVALCYSTHRALAGLYSERARGALATYRILEHISSERLYSVALPHEASPGLYVALILEGLUARGVALALALYSILEALAALASERLEULYSILEGLYPROGLYMETASPPROALATHRGLNILEGLYPROLEUVALSERALALYSGLNARGGLUARGVALCYSGLYTYRILEASPSERGLYPHEGLYGLUGLYALAARGALAALAALAGLYGLYARGALAILEASPGLYPROGLYPHEPHEVALGLUPROTHRVALLEUVALASPTHRTHRGLNALAMETARGVALVALARGGLUGLUILEPHEGLYPROVALLEUVALALAMETPROPHEASPASPVALASPTHRALAVALGLNLEUALAASNASPTHRPROTYRGLYLEUGLYALASERILETRPSERASNASPLEUSERALAILEHISLYSLEUVALPROARGILEALAALAGLYTHRVALTRPVALASNCYSHISSERLEULEUASPASNALALEUPROPHEGLYGLYMETLYSGLNSERGLYPHEGLYARGGLULEUGLYARGALAVALILEASPGLNTYRTHRGLUSERLYSSERVALMETMETASNTYRALA

SEQ ID NO: 2 - protein, 1422 aa, Salmonella enterica
METGLNTYRGLNLEULEUILEASNGLYVALLEUVALASPGLYGLUGLYGLUARGGLNSERVALTYRASNPROALATHRGLYGLUVALILELEUGLUILEALAGLUALASERPROALAGLNVALASPALAALAVALGLNALAALAASPASNALAPHEALAGLUTRPGLYGLNTHRTHRPROLYSALAARGALAGLUCYSLEULEULYSLEUALAASPSERILEGLUGLNASNALALEUGLUPHEALAARGLEUGLUSERGLNASNCYSGLYLYSPROLEUHISCYSVALILEASNASPGLUILEPROALAILEVALASPVALPHEARGPHEPHEALAGLYALAALAARGCYSLEUSERGLYLEUALAALAGLYGLUTYRLEUGLUGLYHISTHRSERMETILEARGARGASPPROILEGLYVALVALALASERILEALAPROTRPASNTYRPROLEUMETMETALAALATRPLYSLEUALAPROALALEUALAALAGLYASNCYSVALVALILELYSPROSERGLUILETHRPROLEUTHRALALEULYSLEUALAVALLEUALALYSASPILEPHEPROPROGLYVALLEUASNVALLEUPHEGLYARGGLYGLNTHRVALGLYASPVALLEUTHRGLYHISGLULYSVALARGMETVALSERLEUTHRGLYSERILEALATHRGLYGLUHISILELEUARGHISTHRALAPROALAILELYSARGTHRHISMETGLULEUGLYGLYLYSALAPROVALILEVALPHEASPASPALAASPLEUASPALAVALALAGLNGLYVALARGTHRPHEGLYPHETYRASNALAGLYGLNASPCYSTHRALAALACYSARGILETYRALAGLNARGGLYILETYRASPALALEUVALGLULYSLEUGLYASNALAVALSERSERLEULYSMETGLYALAPROGLUASPGLUSERTHRGLULEUGLYPROLEUSERSERLEUALAHISLEULYSARGVALTHRALAALAVALGLUGLUALALYSALALEUSERHISILEARGVALILETHRGLYGLYSERGLNTHRGLUGLYLYSGLYTYRTYRPHEALAPROTHRLEULEUALAASPALALYSGLNGLUASPALAILEVALGLNARGGLUVALPHEGLYPROVALVALSERILETHRVALPHEASPASPGLUASPGLNVALLEUARGTRPALAASNASPSERARGTYRGLYLEUALASERSERVALTRPTHRGLNASPVALGLYARGALAHISARGLEUSERALAARGLEUGLNTYRGLYCYSTHRTRPILEASNTHRHISPHEMETLEUVALSERGLUMETPROHISGLYGLYGLNLYSGLNSERGLYTYRGLYLYSASPMETSERLEUTYRGLYLEUGLUASPTYRTHRLEUVALARGHISILEMETVALLYSHIS

SEQ ID NO: 3 - protein, 1425 aa, Thermus thermophilus
METASPTHRGLNLEULEUILEGLYSERARGPHEGLUALAGLYTHRGLUALAGLUGLUHISILELEUASNPROARGTHRGLYALAGLYILEILEASPLEUALAGLUALASERHISALAGLNILEASPALAALAVALASPALAALAGLUARGALAPHEVALGLYTRPSERGLNTHRTHRPROALAGLUARGSERASNALALEULEULYSILEALAASPALAILEGLULYSGLUALAASPGLUPHEALAALALEUGLUALALEUASNCYSGLYLYSPROILEASNALAVALLYSASNASPGLULEUPROALAILEILEASPCYSTRPARGPHEPHEALAGLYALAVALARGASNLEUHISALAPROALAALAGLYGLUTYRLEUPROGLYHISTHRSERMETILEARGARGASPPROILEGLYILEVALGLYSERILEALAPROTRPASNTYRPROLEUMETMETMETALATRPLYSLEUALAPROALAILEGLYGLYGLYASNTHRVALVALPHELYSPROSERGLUGLNTHRPROLEUTHRALALEULYSLEUALAARGLEUILEALAASPILELEUPROGLUGLYVALVALASNVALILETHRGLYARGGLYGLUTHRVALGLYASNALALEUILEASNHISPROLYSVALGLYMETVALSERILETHRGLYASPILEALATHRGLYLYSLYSVALLEUALAALAALAALALYSTHRVALLYSARGTHRHISLEUGLULEUGLYGLYLYSALAPROVALILEVALTYRGLYASPALAASPLEUGLUALAVALVALASNGLYILEARGTHRPHEGLYTYRTYRASNALAGLYGLNASPCYSTHRALAALACYSARGILETYRALAGLUALAGLYILETYRGLULYSLEUVALALAASPLEUTHRSERALAVALSERTHRILEARGTYRASNLEUASPASPASPTHRGLUASNGLUILEGLYPROLEUILESERARGARGGLNARGASPARGVALALASERPHEVALGLUARGALAALAASPGLNLYSHISILEGLUILETHRTHRGLYGLYARGTHRGLYSERASPGLUGLYPHEPHEPHEGLNPROTHRVALVALALAGLYALATHRGLNGLUASPGLUILEVALARGARGGLUVALPHEGLYPROVALVALSERVALTHRARGPHETHRGLYLYSASPASPALAVALALATRPALAASNASPSERASPTYRGLYLEUALASERSERVALTRPTHRLYSASPILESERLYSALAMETARGALAALASERARGLEUGLNTYRGLYCYSTHRTRPILEASNTHRHISPHEMETLEUTHRASNGLUMETPROHISGLYGLYILELYSGLNSERGLYTYRGLYLYSASPMETSERVALTYRALALEUGLUASPTYRTHRALAVALARGHISILEMETILEASNHISGLY

SEQ ID NO: 4 - protein, 1545 aa, Sinorhizobium meliloti
METARGTYRALAASPARGVALALAGLYILESERTRPGLUTHRILEGLUGLUVALARGARGARGLEULYSGLUARGPROALALEUHISPHEILEALAGLYGLUPHEVALPROSERGLUSERGLYGLUTHRPHEPROSERLEUASPPROALATHRASNGLUVALLEUGLYVALALAALAARGGLYGLYGLUARGGLUVALASPARGALAALALYSALAALAHISGLUALAPHEGLNARGTRPSERARGTHRLYSALALYSGLUARGLYSARGTYRLEULEUARGILEALAGLULEUILEGLULYSHISALAASPGLULEUALAVALMETGLUCYSLEUASPALAGLYGLNVALLEUARGILEVALARGALAGLNVALALAARGALAALAGLUASNPHEALAPHETYRALAGLUTYRALAGLUHISALAMETGLUASPARGTHRPHEPROVALASPARGASPTRPLEUTYRTYRTHRVALARGVALPROALAGLYPROVALGLYILEILETHRPROTRPASNALAPROLEUMETLEUSERTHRTRPARGILEALAPROALALEUALAPHEGLYASNTHRVALVALLEULYSPROALAGLUTRPSERPROPHETHRALATHRLYSLEUALAGLUILELEULYSGLUALAASPLEUPROPROGLYVALPHEASNLEUVALGLNGLYPHEGLYGLUGLUALAGLYALAALALEUVALALAHISPROLEUVALPROLEULEUTHRLEUTHRGLYGLUTHRGLUTHRGLYLYSILEVALMETARGASNALAALAASPHISLEULYSARGLEUSERPROGLULEUGLYGLYLYSSERPROALALEUVALPHEALAASPALAASPLEUGLUARGALALEUASPALAVALVALPHEGLNILEPHESERPHEASNGLYGLUARGCYSTHRALASERSERARGLEULEUVALGLUGLULYSILEPHEGLUASPPHEVALGLYLYSVALVALGLUARGALAARGALAILEARGVALGLYHISPROLEUASPPROGLUTHRGLUVALGLYPROLEUILEHISPROGLUHISLEUGLNARGVALLEUGLYTYRVALGLUALAGLYLYSARGGLUGLYALAARGLEULEUVALGLYGLYGLUARGALALYSTHRSERPHEARGGLYGLUASPLEUSERARGGLYASNTYRLEULEUPROTHRVALPHEVALGLYGLUASNHISMETLYSILEALAGLNGLUGLUILEPHEGLYPROVALLEUVALALAILEPROPHELYSASPGLUGLUGLUALALEUARGLYSALAASNASPTHRLYSTYRGLYLEUALAALATYRVALPHETHRARGASPLEUGLUARGALAHISARGLEUALALEUGLULEUGLUALAGLYMETVALTYRLEUASNSERHISASNVALARGHISLEUPROTHRPROPHEGLYGLYVALLYSGLYSERGLYASPARGARGGLUGLYGLYTHRTYRALALEUASPPHETYRTHRASPLEULYSTHRILEALALEUPROLEUARGPROPROHISVALPROLYSPHEGLYLYS

SEQ ID NO: 5 - protein, 1476 aa, Klebsiella pneumoniae
METSERARGMETALAGLUGLNGLNLEUTYRILEASNGLYGLYTYRTHRSERALATHRSERGLYARGTHRPHEGLUTHRILEASNPROALATHRGLYGLUVALLEUALATHRVALGLNALAALAGLYARGGLUASPVALASPARGALAVALGLUSERALAGLNARGGLYGLNLYSILETRPALAALAMETTHRALAMETGLUARGSERARGILELEUARGARGALAVALASPLEULEUARGGLNARGASNASPGLULEUALAARGLEUGLUTHRLEUASPTHRGLYLYSPROLEUSERGLUTHRALAALAVALASPILEVALTHRGLYALAASPVALLEUGLUTYRTYRALAGLYLEUILEPROALALEUGLUGLYSERGLNILEPROLEUARGASPSERSERPHEVALTYRTHRARGARGGLUPROLEUGLYVALVALALAGLYILEGLYALATRPASNTYRPROILEGLNILEALALEUTRPLYSSERALAPROALALEUALAALAGLYASNALAMETILEPHELYSPROSERGLUVALTHRPROLEUTHRALALEULYSLEUALAGLUILETYRSERGLUALAGLYLEUPROASPGLYVALPHEASNVALLEUPROGLYILEGLYALAGLUTHRGLYGLNTYRLEUTHRGLUHISPROASPILEALALYSILESERPHETHRGLYGLYVALALASERGLYLYSLYSVALMETALAASNSERALAALASERSERLEULYSGLUVALTHRMETGLULEUGLYGLYLYSSERPROLEUILEILEALAASPASPALAASPLEUASPLEUALAALAASPILEALAMETMETALAASNPHETYRSERSERGLYGLNVALCYSTHRASNGLYTHRARGVALPHEVALPROALALYSARGLYSALAGLUPHEGLUHISLYSILELEUGLUARGVALALAARGILEARGALAGLYASPLEUPHEALAASPASPTHRASNPHEGLYPROLEUVALSERPHEPROHISARGASPASNVALLEUARGTYRILEGLUSERGLYLYSARGGLUGLYALAARGLEULEUCYSGLYGLYGLUALALEULYSGLYASPGLYPHEASPASNGLYALATRPVALALAPROTHRVALPHETHRASPCYSSERASPGLUMETTHRILEVALARGGLUGLUILEPHEGLYPROVALMETSERILELEUSERTYRALAASPGLUALAGLUVALILEARGARGALAASNALATHRGLUTYRGLYLEUALAALAGLYVALVALTHRPROASNLEUASNARGALAHISARGILEILEHISGLNLEUGLUALAGLYILECYSTRPILEASNSERTRPGLYGLUSERPROALAGLUMETPROVALGLYGLYTYRLYSHISSERGLYILEGLYARGGLUASNGLYVALMETTHRLEUGLNSERTYRTHRGLNVALLYSSERILEGLNVALGLUMETGLYLYSPHEGLNSERILEPHELEUGLU

SEQ ID NO: 6 - protein, 1470 aa, Pseudomonas aeruginosa
METALAARGPHEGLUGLUGLNLYSLEUTYRILEGLYGLYARGTYRVALGLUALASERSERGLYALATHRPHEGLUTHRILEASNPROALAASNGLYGLUVALLEUALALYSVALGLNARGALASERARGGLYASPVALGLUARGALAVALGLNSERALAVALGLUGLYGLNLYSVALTRPALAALAMETTHRALAMETGLNARGSERARGILELEUARGARGALAVALASPILELEUARGGLUARGASNASPGLULEUALAALALEUGLUTHRLEUASPTHRGLYLYSPROLEUALAGLUTHRARGSERVALASPILEVALTHRGLYALAASPVALLEUGLUTYRTYRALAGLYLEUVALPROALAILEGLUGLYGLUGLNILEPROLEUARGGLUTHRSERPHEVALTYRTHRARGARGGLUPROLEUGLYVALVALALAGLYILEGLYALATRPASNTYRPROVALGLNILEALALEUTRPLYSSERALAPROALALEUALAALAGLYASNALAMETILEPHELYSPROSERGLUVALTHRPROLEUTHRALALEULYSLEUALAGLUILETYRTHRGLUALAGLYVALPROASPGLYVALPHEASNVALLEUTHRGLYSERGLYARGGLUVALGLYGLNTRPLEUTHRGLUHISPROLEUILEGLULYSILESERPHETHRGLYGLYTHRSERTHRGLYLYSLYSVALMETALASERALASERSERSERSERLEULYSGLUVALTHRMETGLULEUGLYGLYLYSSERPROLEUILEILEPHEPROASPALAASPLEUASPARGALAALAASPILEALAVALMETALAASNPHEPHESERSERGLYGLNVALCYSTHRASNGLYTHRARGVALPHEILEHISARGSERGLNGLNALAARGPHEGLUALALYSVALLEUGLUARGVALGLNARGILEARGLEUGLYASPPROGLNASPGLUASNTHRASNPHEGLYPROLEUVALSERPHEPROHISMETGLUSERVALLEUGLYTYRILEGLUSERGLYLYSALAGLNLYSALAARGLEULEUCYSGLYGLYGLUARGVALTHRASPGLYALAPHEGLYLYSGLYALATYRVALALAPROTHRVALPHETHRASPCYSARGASPASPMETTHRILEVALARGGLUGLUILEPHEGLYPROVALMETSERILELEUVALTYRASPASPGLUASPGLUALAILEARGARGALAASNASPTHRGLUTYRGLYLEUALAALAGLYVALVALTHRGLNASPLEUALAARGALAHISARGALAILEHISARGLEUGLUALAGLYILECYSTRPILEASNTHRTRPGLYGLUSERPROALAGLUMETPROVALGLYGLYTYRLYSGLNSERGLYVALGLYARGGLUASNGLYLEUTHRTHRLEUALAHISTYRTHRARGILELYSSERVALGLNVALGLULEUGLYASPTYRALASERVALPHE

SEQ ID NO: 7 - protein, 1482 aa, Bacillus cereus
METLEULYSTHRASNILEGLULEULYSPROLYSVALGLUALAPHELEUASNGLUGLUILELYSMETPHEILEASNGLYGLUPHEVALSERALAILEGLYGLYLYSTHRPHEGLUTHRTYRASNPROALATHRGLUASPVALLEUALAVALVALCYSGLUALAGLNGLUGLUASPILEASPALAALAVALLYSALAALAARGSERALAPHEGLUSERGLYPROTRPALAGLUMETTHRTHRALAGLUARGALAHISLEUILETYRLYSLEUALAASPLEUILEGLUGLUHISARGGLUGLULEUALAGLNLEUGLUALALEUASPASNGLYLYSPROTYRGLNVALALALEUASPASPASPILESERALATHRVALGLUASNTYRARGTYRTYRALAGLYTRPTHRTHRLYSILEILEGLYGLNTHRILEPROILESERLYSASPTYRLEUASNTYRTHRARGHISGLUPROVALGLYVALVALGLYGLNILEILEPROTRPASNPHEPROLEUVALMETSERSERTRPLYSMETGLYALAALALEUALATHRGLYCYSTHRILEVALLEULYSPROALASERGLNTHRPROLEUSERLEULEUTYRALAALALYSLEUPHELYSGLUALAGLYPHEPROASNGLYVALVALASNPHEVALPROGLYPHEGLYPROGLUALAGLYALAALAILEVALASNHISHISASPILEASPLYSVALALAPHETHRGLYSERTHRVALTHRGLYLYSTYRILEMETARGGLNSERALAGLUMETILELYSHISVALTHRLEUGLULEUGLYGLYLYSSERPROASNILEILELEUGLUASPALAASPLEUGLUGLUALAILEASNGLYALAPHEGLNGLYILEMETTYRASNHISGLYGLNASNCYSSERALAGLYSERARGVALPHEVALHISARGLYSHISTYRGLUTHRVALVALASPALALEUVALLYSMETALAASNASNVALLYSLEUGLYALAGLYMETGLULYSGLUTHRGLUMETGLYPROLEUVALSERLYSLYSGLNGLNGLUARGVALLEUASNTYRILEGLUGLNGLYLYSLYSGLUGLYALATHRVALALAALAGLYGLYGLUARGALALEUGLULYSGLYTYRPHEVALLYSPROTHRVALPHETHRASPVALTHRASPASPMETTHRILEVALLYSGLUGLUILEPHEGLYPROVALVALVALVALLEUPROPHEASPSERTHRGLUGLUVALILEGLUARGALAASNASNSERSERTYRGLYLEUALAALAGLYVALTRPTHRGLNASNILELYSTHRGLYHISGLNVALALAASNLYSLEULYSALAGLYTHRVALTRPILEASNASPTYRASNLEUGLUASNALAALAALAPROPHEGLYGLYTYRLYSGLNSERGLYILEGLYARGGLULEUGLYSERTYRALALEUASPASNTYRTHRGLUVALLYSSERVALTRPVALASNILELYS

SEQ ID NO: 8 - protein, 1467 aa, Burkholderia pseudomallei
METSERVALTYRGLYLEUGLNARGLEUTYRILEALAGLYALAHISALAASPALATHRSERGLYLYSTHRPHEASPTHRPHEASPPROALATHRGLYGLULEULEUALAARGVALGLNGLNALASERALAASPASPVALASPARGALAVALALASERALAARGGLUGLYGLNARGGLUTRPALAALAMETTHRALAMETGLNARGSERARGILELEUARGARGALAVALGLULEULEUARGGLUARGASNASPALALEUALAGLULEUGLUMETARGASPTHRGLYLYSPROILEALAGLUTHRARGALAVALASPILEVALTHRGLYALAASPVALILEGLUTYRTYRALAGLYLEUALATHRALAILEGLUGLYLEUGLNVALPROLEUARGPROGLUSERPHEVALTYRTHRARGARGGLUPROLEUGLYVALCYSALAGLYILEGLYALATRPASNTYRPROILEGLNILEALACYSTRPLYSSERALAPROALALEUALAALAGLYASNALAMETILEPHELYSPROSERGLUVALTHRPROLEUSERALALEULYSLEUALAGLUILETYRTHRGLUALAGLYVALPROALAGLYVALPHEASNVALVALGLNGLYASPGLYSERVALGLYALALEULEUSERALAHISPROGLYILEALALYSVALSERPHETHRGLYGLYVALGLUTHRGLYLYSLYSVALMETSERLEUALAGLYALASERSERLEULYSGLUVALTHRMETGLULEUGLYGLYLYSSERPROLEUILEVALPHEASPASPALAASPLEUASPARGALAALAASPILEALAVALTHRALAASNPHEPHESERALAGLYGLNVALCYSTHRASNGLYTHRARGVALPHEVALGLNGLNALAVALLYSASPALAPHEVALGLUARGVALLEUALAARGVALALAARGILEARGVALGLYLYSPROSERASPSERASPTHRASNPHEGLYPROLEUALASERALAALAGLNLEUASPLYSVALLEUGLYTYRILEASPSERGLYLYSALAGLUGLYALALYSLEULEUALAGLYGLYALAARGLEUVALASNASPHISPHEALASERGLYGLNTYRVALALAPROTHRVALPHEGLYASPCYSARGASPASPMETARGILEVALARGGLUGLUILEPHEGLYPROVALMETSERILELEUSERPHEGLUTHRGLUASPGLUALAILEALAARGALAASNALATHRASPTYRGLYLEUALAALAGLYVALVALTHRGLUASNLEUSERARGALAHISARGALAILEHISARGLEUGLUALAGLYILECYSTRPILEASNTHRTRPGLYGLUSERPROALAGLUMETPROVALGLYGLYTYRLYSGLNSERGLYVALGLYARGGLUASNGLYILETHRTHRLEUGLUHISTYRTHRARGILELYSSERVALGLNVALGLULEUGLYARGTYRGLNPROVALPHE

SEQ ID NO: 9 - protein, 1479 aa, Agrobacterium tumefaciens
METTHRILEALATHRPROLEULYSALAGLNPROLYSALASERHISPHEILEASPGLYASPTYRVALGLUASPASNTHRGLYTHRPROPHEGLUSERILEPHEPROALATHRGLYGLUMETILEALALYSLEUHISALAALATHRPROALAILEVALGLUARGALAILEALASERALALYSARGALAGLNLYSGLUTRPALAALAMETSERPROMETALAARGGLYARGILELEULYSARGALAALAASPILEMETARGGLUARGASNASPALALEUSERTHRLEUGLUTHRLEUASPTHRGLYLYSPROILEGLNGLUTHRILEVALALAASPPROTHRSERGLYALAASPALAPHEGLUPHEPHEGLYGLYILEALAPROSERALALEUASNGLYASPTYRILEPROLEUGLYGLYASPPHEALATYRTHRLYSARGVALPROLEUGLYVALCYSVALGLYILEGLYALATRPASNTYRPROGLNGLNILEALACYSTRPLYSALAALAPROALALEUVALALAGLYASNALAMETVALPHELYSPROSERGLUASNTHRPROLEUGLYALALEULYSILEALAGLUILELEUILEGLUALAGLYLEUPROLYSGLYLEUPHEASNVALILEGLNGLYASPARGASPTHRGLYPROLEULEUVALASNHISPROASPVALALALYSVALSERLEUTHRGLYSERVALPROTHRGLYARGLYSVALALAALAALAALAALAGLYHISLEULYSHISVALTHRMETGLULEUGLYGLYLYSSERPROMETILEVALPHEASPASPALAASPILEGLUSERALAVALGLYGLYALAMETLEUGLYASNPHETYRSERSERGLYGLNVALCYSSERASNGLYTHRARGVALPHEVALGLNLYSLYSALALYSALAARGPHELEUGLUASNLEULYSARGARGTHRGLUALAMETILELEUGLYASPPROLEUASPTYRALATHRHISLEUGLYPROLEUVALSERLYSALAGLNGLNGLULYSVALLEUSERTYRILEGLULYSGLYLYSALAGLUGLYALATHRLEUILETHRGLYGLYGLYILEPROASNASNVALALAGLYGLUGLYALATYRVALGLNPROTHRVALPHEALAASPVALTHRASPASPMETTHRILEALAARGGLUGLUILEPHEGLYPROVALMETCYSVALLEUASPPHEASPASPGLUASPGLUVALLEUALAARGALAASNALATHRGLUPHEGLYLEUALAGLYGLYVALPHETHRALAASPLEUALAARGALAHISARGVALVALASPGLYLEUGLUALAGLYTHRLEUTRPILEASNTHRTYRASNLEUCYSPROVALGLUILEPROPHEGLYGLYSERLYSGLNSERGLYPHEGLYARGGLUASNSERALAALAALALEUGLUHISTYRSERGLULEULYSTHRVALTYRVALSERTHRGLYLYSVALASPALAPROTYR

SEQ ID NO: 10 - protein, 1470 aa, Pseudomonas aeruginosa
METALAARGPHEGLUGLUGLNLYSLEUTYRILEGLYGLYARGTYRVALGLUALASERSERGLYALATHRPHEGLUTHRILEASNPROALAASNGLYGLUVALLEUALALYSVALGLNARGALASERARGGLUASPVALGLUARGALAVALGLNSERALAVALGLUGLYGLNLYSVALTRPALAALAMETTHRALAMETGLNARGSERARGILELEUARGARGALAVALASPILELEUARGGLUARGASNASPGLULEUALAALALEUGLUTHRLEUASPTHRGLYLYSPROLEUALAGLUTHRARGSERVALASPILEVALTHRGLYALAASPVALLEUGLUTYRTYRALAGLYLEUVALPROALAILEGLUGLYGLUGLNILEPROLEUARGGLUTHRSERPHEVALTYRTHRARGARGGLUPROLEUGLYVALVALALAGLYILEGLYALATRPASNTYRPROVALGLNILEALALEUTRPLYSSERALAPROALALEUALAALAGLYASNALAMETILEPHELYSPROSERGLUVALTHRPROLEUTHRALALEULYSLEUALAGLUILETYRTHRGLUALAGLYVALPROASPGLYVALPHEASNVALLEUTHRGLYSERGLYARGGLUVALGLYGLNTRPLEUTHRGLUHISPROLEUILEGLULYSILESERPHETHRGLYGLYTHRSERTHRGLYLYSLYSVALMETALASERALASERSERSERSERLEULYSGLUVALTHRMETGLULEUGLYGLYLYSSERPROLEUILEILEPHEPROASPALAASPLEUASPARGALAALAASPILEALAVALMETALAASNPHEPHESERSERGLYGLNVALCYSTHRASNGLYTHRARGVALPHEILEHISARGSERGLNGLNALAARGPHEGLUALALYSVALLEUGLUARGVALGLNARGILEARGLEUGLYASPPROGLNASPGLUASNTHRASNPHEGLYPROLEUVALSERPHEPROHISMETGLUSERVALLEUGLYTYRILEGLUSERGLYLYSALAGLNLYSALAARGLEULEUCYSGLYGLYGLUARGVALTHRASPGLYALAPHEGLYLYSGLYALATYRVALALAPROTHRVALPHETHRASPCYSARGASPASPMETTHRILEVALARGGLUGLUILEPHEGLYPROVALMETSERILELEUVALTYRASPASPGLUASPGLUALAILEARGARGALAASNASPTHRGLUTYRGLYLEUALAALAGLYVALVALTHRGLNASPLEUALAARGALAHISARGALAILEHISARGLEUGLUALAGLYILECYSTRPILEASNTHRTRPGLYGLUSERPROALAGLUMETPROVALGLYGLYTYRLYSGLNSERGLYVALGLYARGGLUASNGLYLEUTHRTHRLEUALAHISTYRTHRARGILELYSSERVALGLNVALGLULEUGLYASPTYRALASERVALPHE

SEQ ID NO: 11 - protein, 1509 aa, Burkholderia cenocepacia
METASNMETGLUTHRASNGLUTHRPHEALALEULEUASPALATHRARGALAPHELEUALALYSPROLYSGLNMETLEUILEGLYALAGLUTRPSERASPALAALASERGLYARGGLNLEUASPVALVALASNPROALAASPGLYTHRVALILEALAARGVALPROGLUALAASPGLUARGASPVALGLNGLNALAVALALAALAALAARGARGALAPHEASPALAGLYPROTRPARGTHRALALYSTHRTHRARGARGGLUARGLEUMETLEUVALLEUALAASPLEUILEGLUALAASNALAARGGLULEUALAGLUILEGLUSERLEUASPASNGLYLYSPROVALMETVALALAGLNGLYLEUASPVALALAMETALAALAGLNCYSPHEARGTYRMETALAGLYTRPALATHRLYSILEGLUGLYSERVALILEASPALAGLYMETPROTYRLEUPROASPSERGLUILEPHEALATYRTHRARGLYSGLUPROVALGLYVALVALGLYALAILEILEPROTRPASNPHEPROLEULEUMETALAALATRPLYSILEALAPROALALEUALATHRGLYCYSTHRVALVALLEULYSPROALAGLUASPTHRPROLEUSERALALEUARGLEUGLYGLULEUILEGLNALAALAGLYPHEPROASPGLYVALVALASNILEVALTHRGLYTYRGLYHISTHRALAGLYALAALALEUSERARGASPPROARGILEASPLYSILEALAPHETHRGLYSERTHRGLNTHRGLYLYSTHRILEGLYHISALAALALEUASPASNMETTHRARGMETSERLEUGLULEUGLYGLYLYSSERPROVALILEVALLEUPROASPVALASPLEUASPLYSALAALAGLNGLYVALALAASNALAILEPHEPHEASNGLNGLYGLNVALCYSTHRALAGLYSERARGALATYRILEHISSERLYSVALPHEASPGLYVALILEGLUARGVALALALYSILEALAALASERLEULYSILEGLYPROGLYMETASPPROALATHRGLNILEGLYPROLEUVALSERALALYSGLNARGGLUARGVALCYSGLYTYRILEASPSERGLYPHEGLYGLUGLYALAARGALAALAALAGLYGLYARGALAILEASPGLYPROGLYPHEPHEVALGLUPROTHRVALLEUVALASPTHRTHRGLNALAMETARGVALVALARGGLUGLUILEPHEGLYPROVALLEUVALALAMETPROPHEASPASPVALASPTHRALAVALGLNLEUALAASNASPTHRPROTYRGLYLEUGLYALASERILETRPSERASNASPLEUSERALAILEHISLYSLEUVALPROARGILEALAALAGLYTHRVALTRPVALASNCYSHISSERLEULEUASPASNALALEUPROPHEGLYGLYMETLYSGLNSERGLYPHEGLYARGARGLEUGLYARGALAVALILEASPGLNTYRTHRGLUSERLYSSERVALMETMETASNTYRALA

SEQ ID NO: 12 - protein, 1509 aa, Burkholderia cenocepacia
METASNMETGLUTHRASNGLUTHRPHEALALEULEUASPALATHRARGALAPHELEUALALYSPROLYSGLNMETLEUILEGLYALAGLUTRPSERASPALAALASERGLYARGGLNLEUASPVALVALASNPROALAASPGLYTHRVALILEALAARGVALPROGLUALAASPGLUARGASPVALGLNGLNALAVALALAALAALAARGARGALAPHEASPALAGLYPROTRPARGTHRALALYSTHRTHRARGARGGLUARGLEUMETLEUVALLEUALAASPLEUILEGLUALAASNALAARGGLULEUALAGLUILEGLUSERLEUASPASNGLYLYSPROVALMETVALALAGLNGLYLEUASPVALALAMETALAALAGLNCYSPHEARGTYRMETALAGLYTRPALATHRLYSILEGLUGLYSERVALILEASPALAGLYMETPROTYRLEUPROASPSERGLUILEPHEALATYRTHRARGLYSGLUPROVALGLYVALVALGLYALAILEILEPROTRPASNPHEPROLEULEUMETALAALATRPLYSILEALAPROALALEUALATHRGLYCYSTHRVALVALLEULYSPROALAGLUASPTHRPROLEUSERALALEUARGLEUGLYGLULEUILEGLNALAALAGLYPHEPROASPGLYVALVALASNILEVALTHRGLYTYRGLYHISTHRALAGLYALAALALEUSERARGASPPROARGILEASPLYSILEALAPHETHRGLYSERTHRGLNTHRGLYLYSTHRILEGLYHISALAALALEUASPASNMETTHRARGMETSERLEUGLULEUGLYGLYLYSSERPROVALILEVALLEUPROASPVALASPLEUASPLYSALAALAGLNGLYVALALAASNALAILEPHEPHEASNGLNGLYGLNVALCYSTHRALAGLYSERARGALATYRILEHISSERLYSVALPHEASPGLYVALILEGLUARGVALALALYSILEALAALASERLEULYSILEGLYPROGLYMETASPPROALATHRGLNILEGLYPROLEUVALSERALALYSGLNARGGLUARGVALCYSGLYTYRILEASPSERGLYPHEGLYGLUGLYALAARGALAALAALAGLYGLYARGALAILEASPGLYPROGLYPHEPHEVALGLUPROTHRVALLEUVALASPTHRTHRGLNALAMETARGVALVALARGGLUGLUILEPHEGLYPROVALLEUVALALAMETPROPHEASPASPVALASPTHRALAVALGLNLEUALAASNASPTHRPROTYRGLYLEUGLYALASERILETRPSERASNASPLEUSERALAILEHISLYSLEUVALPROARGILEALAALAGLYTHRVALTRPVALASNCYSHISSERLEULEUASPASNALALEUPROPHEGLYGLYMETLYSGLNSERGLYPHEGLYARGGLULEUGLYARGALAVALILEASPGLNTYRTHRGLUSERLYSSERVALMETMETASNTYRALA

SEQ ID NO: 13 - protein, 1509 aa, Burkholderia cenocepacia
METASNMETGLUTHRASNGLUTHRPHEALALEULEUASPALATHRARGALAPHELEUALALYSPROLYSGLNMETLEUILEGLYALAGLUTRPSERASPALAALASERGLYARGGLNLEUASPVALVALASNPROALAASPGLYTHRVALILEALAARGVALPROGLUALAASPGLUARGASPVALGLNGLNALAVALALAALAALAARGARGALAPHEASPALAGLYPROTRPARGTHRALALYSTHRTHRASPARGGLUARGLEUMETLEUVALLEUALAASPLEUILEGLUALAASNALAARGGLULEUALAGLUILEGLUSERLEUASPASNGLYLYSPROVALMETVALALAGLNGLYLEUASPVALALAMETALAALAGLNCYSPHEARGTYRMETALAGLYTRPALATHRLYSILEGLUGLYSERVALILEASPALAGLYMETPROTYRLEUPROASPSERGLUILEPHEALATYRTHRARGLYSGLUPROVALGLYVALVALGLYALAILEILEPROTRPASNPHEPROLEULEUMETALAALATRPLYSILEALAPROALALEUALATHRGLYCYSTHRVALVALLEULYSPROALAGLUASPTHRPROLEUSERALALEUARGLEUGLYGLULEUILEGLNALAALAGLYPHEPROASPGLYVALVALASNILEVALTHRGLYTYRGLYHISTHRALAGLYALAALALEUSERARGASPPROARGILEASPLYSILEALAPHETHRGLYSERTHRGLNTHRGLYLYSTHRILEGLYHISALAALALEUASPASNMETTHRARGMETSERLEUGLULEUGLYGLYLYSSERPROVALILEVALLEUPROASPVALASPLEUASPLYSALAALAGLNGLYVALALAASNALAILEPHEPHEASNGLNGLYGLNVALCYSTHRALAGLYSERARGALATYRILEHISSERLYSVALPHEASPGLYVALILEGLUARGVALALALYSILEALAALASERLEULYSILEGLYPROGLYMETASPPROALATHRGLNILEGLYPROLEUVALSERALALYSGLNARGGLUARGVALCYSGLYTYRILEASPSERGLYPHEGLYGLUGLYALAARGALAALAALAGLYGLYARGALAILEASPGLYPROGLYPHEPHEVALGLUPROTHRVALLEUVALASPTHRTHRGLNALAMETARGVALVALARGGLUGLUILEPHEGLYPROVALLEUVALALAMETPROPHEASPASPVALASPTHRALAVALGLNLEUALAASNASPTHRPROTYRGLYLEUGLYALASERILETRPSERASNASPLEUSERALAILEHISLYSLEUVALPROARGILEALAALAGLYTHRVALTRPVALASNCYSHISSERLEULEUASPASNALALEUPROPHEGLYGLYMETLYSGLNSERGLYPHEGLYARGARGLEUGLYARGALAVALILEASPGLNTYRTHRGLUSERLYSSERVALMETMETASNTYRALA

SEQ ID NO: 14 - protein, 1509 aa, Burkholderia cenocepacia
METASNMETGLUTHRASNGLUTHRPHEALALEULEUASPALATHRARGALAPHELEUALALYSPROLYSGLNMETLEUILEGLYALAGLUTRPSERASPALAALASERGLYARGGLNLEUASPVALVALASNPROALAASPGLYTHRVALILEALAARGVALPROGLUALAASPGLUARGASPVALGLNGLNALAVALALAALAALAARGARGALAPHEASPALAGLYPROTRPARGTHRALALYSTHRTHRASPARGGLUARGLEUMETLEUVALLEUALAASPLEUILEGLUALAASNALAARGGLULEUALAGLUILEGLUSERLEUASPASNGLYLYSPROVALMETVALALAGLNGLYLEUASPVALALAMETALAALAGLNCYSPHEARGTYRMETALAGLYTRPALATHRLYSILEGLUGLYSERVALILEASPALAGLYMETPROTYRLEUPROASPSERGLUILEPHEALATYRTHRARGLYSGLUPROVALGLYVALVALGLYALAILEILEPROTRPASNPHEPROLEULEUMETALAALATRPLYSILEALAPROALALEUALATHRGLYCYSTHRVALVALLEULYSPROALAGLUASPTHRPROLEUSERALALEUARGLEUGLYGLULEUILEGLNALAALAGLYPHEPROASPGLYVALVALASNILEVALTHRGLYTYRGLYHISTHRALAGLYALAALALEUSERARGASPPROARGILEASPLYSILEALAPHETHRGLYSERTHRGLNTHRGLYLYSTHRILEGLYHISALAALALEUASPASNMETTHRARGMETSERLEUGLULEUGLYGLYLYSSERPROVALILEVALLEUPROASPVALASPLEUASPLYSALAALAGLNGLYVALALAASNALAILEPHEPHEASNGLNGLYGLNVALCYSTHRALAGLYSERARGALATYRILEHISSERLYSVALPHEASPGLYVALILEGLUARGVALALALYSILEALAALASERLEULYSILEGLYPROGLYMETASPPROALATHRGLNILEGLYPROLEUVALSERALALYSGLNARGGLUARGVALCYSGLYTYRILEASPSERGLYPHEGLYGLUGLYALAARGALAALAALAGLYGLYARGALAILEASPGLYPROGLYPHEPHEVALGLUPROTHRVALLEUVALASPTHRTHRGLNALAMETARGVALVALARGGLUGLUILEPHEGLYPROVALLEUVALALAMETPROPHEASPASPVALASPTHRALAVALGLNLEUALAASNASPTHRPROTYRGLYLEUGLYALASERILETRPSERASNASPLEUSERALAILEHISLYSLEUVALPROARGILEALAALAGLYTHRVALTRPVALASNCYSHISSERLEULEUASPASNALALEUPROPHEGLYGLYMETLYSGLNSERGLYPHEGLYARGGLULEUGLYARGALAVALILEARGGLNTYRTHRGLUSERLYSSERVALMETMETASNTYRALA

SEQ ID NO: 15 - protein, 1509 aa, Burkholderia cenocepacia
METASNMETGLUTHRASNGLUTHRPHEALALEULEUASPALATHRARGALAPHELEUALALYSPROLYSGLNMETLEUILEGLYALAGLUTRPSERASPALAALASERGLYARGGLNLEUASPVALVALASNPROALAASPGLYTHRVALILEALAARGVALPROGLUALAASPGLUARGASPVALGLNGLNALAVALALAALAALAARGARGALAPHEASPALAGLYPROTRPARGTHRALALYSTHRTHRASPARGGLUARGLEUMETLEUVALLEUALAASPLEUILEGLUALAASNALAARGGLULEUALAGLUILEGLUSERLEUASPASNGLYLYSPROVALMETVALALAGLNGLYLEUASPVALALAMETALAALAGLNCYSPHEARGTYRMETALAGLYTRPALATHRLYSILEGLUGLYSERVALILEASPALAGLYMETPROTYRLEUPROASPSERGLUILEPHEALATYRTHRARGLYSGLUPROVALGLYVALVALGLYALAILEILEPROTRPASNPHEPROLEULEUMETALAALATRPLYSILEALAPROALALEUALATHRGLYCYSTHRVALVALLEULYSPROALAGLUASPTHRPROLEUSERALALEUARGLEUGLYGLULEUILEGLNALAALAGLYPHEPROASPGLYVALVALASNILEVALTHRGLYTYRGLYHISTHRALAGLYALAALALEUSERARGASPPROARGILEASPLYSILEALAPHETHRGLYSERTHRGLNTHRGLYLYSTHRILEGLYHISALAALALEUASPASNMETTHRARGMETSERLEUGLULEUGLYGLYLYSSERPROVALILEVALLEUPROASPVALASPLEUASPLYSALAALAGLNGLYVALALAASNALAILEPHEPHEASNGLNGLYGLNVALCYSTHRALAGLYSERARGALATYRILEHISSERLYSVALPHEASPGLYVALILEGLUARGVALALALYSILEALAALASERLEULYSILEGLYPROGLYMETASPPROALATHRGLNILEGLYPROLEUVALSERALALYSGLNARGGLUARGVALCYSGLYTYRILEASPSERGLYPHEGLYGLUGLYALAARGALAALAALAGLYGLYARGALAILEASPGLYPROGLYPHEPHEVALGLUPROTHRVALLEUVALASPTHRTHRGLNALAMETARGVALVALARGGLUGLUILEPHEGLYPROVALLEUVALALAMETPROPHEASPASPVALASPTHRALAVALGLNLEUALAASNASPTHRPROTYRGLYLEUGLYALASERILETRPSERASNASPLEUSERALAILEHISLYSLEUVALPROARGILEALAALAGLYTHRVALTRPVALASNCYSHISSERLEULEUASPASNALALEUPROPHEGLYGLYMETLYSGLNSERGLYPHEGLYARGGLULEUGLYARGALAVALILEASPGLNTYRTHRARGSERLYSSERVALMETMETASNTYRALA